(19) United States
(12) Patent Application Publication
(10) Pub. No.: US 2024/0181178 A1
(43) Pub. Date: Jun. 6, 2024

(54) VIBRATIONAL POWDER APPLICATOR

(71) Applicant: Davol Inc., Warwick, RI (US)

(72) Inventors: Jonathan Bruce Trexler, Rehoboth, MA (US); Nathan Stewart Cauldwell, Hope, RI (US); Oliver Sowerby, Cambridge (GB); Steve McLellan, Cambridge (GB); Tom Etheridge, Cambridge (GB); Michael Herd, Cambridge (GB); Julia Allwright, Cambridge (GB); Christopher Bowley, Newport, RI (US)

(73) Assignee: Davol Inc., Warwick, RI (US)

(21) Appl. No.: 18/284,766

(22) PCT Filed: Mar. 31, 2021
(86) PCT No.: PCT/US2021/025139

§ 371 Date: Sep. 28, 2023

(51) Int. Cl. A61M 13/00; B05B 1/34; B05B 7/12; B05B 7/14

(52) U.S. Cl. CPC A61M 13/00; B05B 1/3402; B05B 7/12; B05B 7/1422; A61M 2202/064; A61M 2206/20

(57) ABSTRACT

Vibrational powder applicators and related methods are disclosed. In some embodiments, a vibrational powder applicator may include a powder storage chamber, a powder (e.g., a therapeutic powder) disposed within the powder storage chamber, an actuator operatively coupled to the powder storage chamber and configured to vibrationally agitate the powder when activated, and an outlet in fluid communication with the powder storage chamber. In some embodiments, a vibrational powder applicator may include a flow restrictor and/or a valve.

### FIELD

Disclosed embodiments are related to powder applicators and associated methods.

### BACKGROUND

Powder applicators are used in many different applications to apply various types of powders to a desired surface, such as in the delivery of therapeutic powders to a desired location of a subject for therapeutic purposes. Powders that are delivered using these applicators tend to be lightweight, low-density powders with a Hausner ratio between 1.00 and 1.18. Some applicators fluidize a powder by directing a flow of gas toward the powder.

### SUMMARY

In some embodiments, a method of applying a therapeutic powder includes positioning an outlet of a powder applicator containing a therapeutic powder below a powder storage chamber of the powder applicator relative to a local direction of gravity, vibrationally agitating the therapeutic powder, and dispensing at least a portion of the therapeutic powder through the outlet of the powder applicator.

In some embodiments, a vibrational powder applicator includes a powder storage chamber, a therapeutic powder disposed within the powder storage chamber, an actuator operatively coupled to the powder storage chamber and configured to vibrationally agitate the therapeutic powder when activated, and an outlet in fluid communication with the powder storage chamber.

In some embodiments, a vibrational powder applicator includes a powder storage chamber configured to contain a powder, an actuator operatively coupled to the powder storage chamber and configured to vibrationally agitate the powder when activated, an outlet in fluid communication with the powder storage chamber, and a flow restrictor disposed between the powder storage chamber and the outlet.

In some embodiments, a vibrational powder applicator includes a powder storage chamber configured to contain a powder, an actuator operatively coupled to the powder storage chamber and configured to vibrationally agitate the powder when activated, an outlet in fluid communication with the powder storage chamber, and a valve disposed between the powder storage chamber and the outlet. The valve is configured to selectively permit or prevent flow of the powder from the powder storage chamber to the outlet.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a cross-sectional schematic of one embodiment of a vibrational powder applicator;

FIG. 2A is a top view of one embodiment of a vibrational powder applicator;

FIG. 2B is a cross-sectional side view of the vibrational powder applicator of FIG. 2A taken along line 2B-2B;

FIG. 3A is a cross-sectional back view of one embodiment of a flow restrictor;

FIG. 3B is a cross-sectional side view of a distal end of one embodiment of a vibrational powder applicator that includes the flow restrictor of FIG. 3A taken along line 3B-3B;

FIG. 4A is a cross-sectional back view of a first embodiment of a flow restrictor with a first number of fins;

FIG. 4B is a cross-sectional back view of a second embodiment of a flow restrictor with a second number of fins;

FIG. 4C is a cross-sectional back view of a third embodiment of a flow restrictor with a third number of fins;

FIG. 4D is a cross-sectional back view of a fourth embodiment of a flow restrictor with a fourth number of fins; and

FIG. 5 is a cross-sectional side view of one embodiment of a vibrational powder applicator that includes a valve.

### DETAILED DESCRIPTION

Therapeutic powders may vary both in particle size and density. These properties impact the flow character, or flowability, of the powder. The Hausner ratio, which is calculated by dividing the measured tapped density of a powder by its bulk density, can be used to assess the flowability of a powder. Generally, the lower the Hausner ratio, the better the flowability. For example, powders with a Hausner ratio between 1.00 and 1.18 may be considered to exhibit excellent to good flow characteristics, whereas powders with a Hausner ratio above 1.18 exhibit fair to poor flow characteristics. There may also be other powder characteristics such as particle morphology, basic flowability energy, aerated energy, aeration ratio, wall friction angle, compression percent, static charge, moisture content, and other appropriate parameters which may be used to characterize the overall flowability of a powder. Particles with poor flowability are relatively difficult to fluidize, which may make them ill-suited for certain application methods.

Hemostatic powders are therapeutic powders that are used to manage or stop bleeding. These powders are commonly applied via applicators. Conventional applicators may use pressurized gas (which may be generated by a manual bellows, or an automated gas flow, for example) in order to fluidize the powder for delivery towards a target area, such as a bleed site. Existing applicators use relatively high pressure and/or high velocity gas, and are generally effective at fluidizing and applying hemostatic powders with relatively small particle sizes and/or relatively low densities. Such hemostatic powders may exhibit Hausner ratios between 1.00 and 1.18. However, these small particle size and/or low-density hemostatic powders may be ineffective at breaking the surface tension of flowing blood, and therefore may not reach a desired location beneath the flowing blood. Accordingly, the use of larger and/or more dense powders capable of breaking the surface tension of flowing blood may be advantageous in certain applications. However, larger and/or more dense powders may be difficult to fluidize using standard pressure-based applicators. Furthermore, using pressurized gas to expel larger and/or more dense powders may result in reduced controllability and limited delivery precision.

In view of the above, the Inventors have recognized the benefits of an improved therapeutic powder applicator capable of focal dispensing of powder (including relatively large and/or dense powder) in a specific delivery area. Such an applicator may be configured to effectively fluidize and apply therapeutic powders in a controlled, targeted, and repeatable fashion. Whereas conventional applicators may be limited to applying hemostatic powders in a relatively coarse and/or imprecise fashion, an improved applicator may be capable of delivering a variety of different types, sizes, and densities of powders in a controllable fashion, and may enable focused delivery of powder to a precise target area in some embodiments.

In view of the above, the Inventors have recognized the benefits of an applicator that may control the delivery of powder from the applicator using vibration. Without wishing to be bound by theory, applying vibrational energy to a powder (e.g., powder that is disposed within an applicator) may be associated with fluidizing the powder such that the powder may flow under the influence of gravity. When an applicator containing a powder is held in certain orientations, applying vibrational energy to the powder may fluidize the powder and allow the powder to flow from a powder storage chamber within the applicator toward a tip of the applicator and out of an outlet of the applicator. Such a vibrational powder applicator may enable controllable, focused, precise, and repeatable delivery of powder.

In some embodiments, a vibrational powder applicator includes a powder storage chamber configured to contain a powder, such as a therapeutic powder (e.g., a hemostatic powder). An actuator may be operatively coupled to the powder storage chamber and configured to vibrationally agitate the powder when activated. An outlet of the applicator may be in fluid communication with the powder storage chamber. In some embodiments, the powder storage chamber and the outlet are configured such that the powder may move from the powder storage chamber toward the outlet when the powder is vibrationally agitated by the actuator. For example, a vibrational powder applicator may include a proximal end and a distal end. The powder storage chamber may be associated with the proximal end, and the outlet may be associated with the distal end. When the applicator is oriented such that the distal end (and the outlet) is below the proximal end (and the storage chamber) relative to a local direction of gravity, vibrationally agitating the powder may fluidize the powder and allow the powder to flow from the storage chamber toward the outlet and through the outlet under the influence of gravity, thereby dispensing at least a portion of the powder.

An actuator of a vibrational powder applicator may include any suitable actuator configured to vibrate powder associated with the applicator. In some embodiments, an actuator may include a motor, such as a brushed motor, a brushless motor, a stepper motor, or any other appropriate type of motor. In some embodiments that include a motor, the motor may be configured to induce vibration in the powder by rotating an eccentric load coupled to the motor. In some embodiments, the actuator may include a linear actuator, such as a linear actuator configured to translate a mass back and forth to vibrate the powder. An actuator of a vibrational powder applicator may be directly coupled to a portion of the applicator, though the actuator may also be coupled to any suitable gearing, transmission, and/or linkage to vibrate the powder, as the disclosure is not so limited. In view of the above, it should be understood that any appropriate type of actuator capable of applying a vibrational force to a portion of the applicator capable of fluidizing the powder contained with the applicator may be used as the disclosure is not limited in this fashion.

It should be appreciated that an actuator of a vibrational powder applicator may be disposed in any suitable location and/or in any suitable orientation, as the disclosure is not limited in this regard. The applicator may include a proximal end and a distal end, and the outlet may be positioned on a distal portion of the applicator. In some embodiments, the actuator may be disposed proximally relative to a distal portion of the powder storage chamber. In some embodiments, the actuator may be disposed distally relative to a proximal portion of the powder storage chamber. In some embodiments, the actuator may be disposed proximal to the outlet. In some embodiments, the actuator may be disposed proximal to a tapered portion of the applicator leading to a nozzle. In some embodiments, the actuator may be contained within an outer casing of the powder applicator, attached to a housing of the applicator, or operatively coupled to a housing of the applicator in any appropriate fashion as the disclosure is not limited in this fashion. The Inventors have appreciated that positioning the actuator on a portion of the applicator that is closer to a distal portion of the powder storage chamber may be advantageous in that such positioning may reduce the amount of residual powder in the storage chamber after use by promoting powder fluidization regardless of how much powder remains in the chamber. Additionally, placement of the motor may be at least partly associated with the speed at which powder is delivered by the applicator. However, it should be appreciated that the present disclosure is not limited to any specific positioning of an actuator relative to any other component or portion of the applicator.

While the above description has at times referred to a single actuator, the present disclosure is not limited regarding the number of actuators included in a vibrational powder applicator. A vibrational powder applicator may include one, two, three, four, five, or any other suitable number of actuators arranged and/or distributed in any suitable fashion. An applicator may include different types of actuators configured to induce vibration. An applicator may include additional applicators not configured to induce vibration, but rather configured to perform another operation related to the delivery of powder.

In some embodiments, an applicator may include a sleeve that surrounds at least a portion of the housing. A sleeve may be configured to at least partially isolate vibration of the powder storage chamber from the user's hand. For example, an elastomeric sleeve may enable the actuator to vibrate the powder storage chamber (or other portion of the applicator) while reducing the amount of vibration experienced by the user. A sleeve material may be selected to dampen vibration from the actuator. For example, a sleeve may be an elastomeric material, a viscoelastic material, a rubber, a silicone, a polyurethane, or any other suitable material. Additionally, a sleeve may provide an ergonomic grip for the user. Other additional vibration isolation components (including but limited to O-rings and gaskets) may be included in an applicator, as the disclosure is not limited in this regard.

In some embodiments, a method of applying a hemostatic powder may include positioning an outlet of a powder applicator containing the hemostatic powder below a powder storage chamber of the powder applicator relative to a local direction of gravity, vibrationally agitating the hemostatic powder, and dispensing at least a portion of the hemostatic powder through the outlet of the powder applicator. The method may also include positioning the outlet of the powder applicator above a target delivery site prior to dispensing the powder. As described above, vibrationally agitating a powder may include activating an actuator, such as activating a motor configured to rotate an eccentric load. In some embodiments, the powder may remain fluidized only while the actuator is activated. Correspondingly, in some embodiments, the method may further include deactivating the actuator to stop dispensing the powder.

Without wishing to be bound by theory, the amount of fluidization of a powder may depend at least in part on the internal geometry of the vessel in which the powder is contained. For example, in the case of a powder applicator, a minimum internal dimension (such as an inside diameter of a nozzle leading to an outlet) may in part determine fluidization behavior of the powder. For example, when holding other variables such as powder size, and powder density constant, a minimum internal dimension (e.g., nozzle inside diameter) that is below a first threshold dimension may prevent powder flow regardless of whether vibration is applied to the powder. For instance, the inside diameter of a nozzle may be too small (relative to the particle size of the powder) for the powder to flow through the nozzle. Similarly, a minimum internal dimension above a second threshold dimension that is greater than the first threshold dimension may permit the free flow of powder through the nozzle regardless of whether vibration is applied to the powder. For example, the inside diameter of a nozzle may be so large (relative to the particle size of the powder) that the powder flows freely through the nozzle simply under the influence of gravity. In some embodiments, where a minimum internal dimension is above the first threshold dimension and below the second threshold dimension, powder flow through the nozzle and outlet of an applicator may occur when vibration is applied (e.g., when the actuator is activated) and powder flow through the nozzle and outlet may be substantially prevented when the vibration is stopped (e.g., when the actuator is deactivated).

Of course, a minimum internal dimension may affect other system parameters, including but not limited to powder flow rate. Without wishing to be bound by theory, a larger minimum internal dimension may be associated with higher flow rates of powder compared to a smaller minimum internal dimension. In certain situations, it may be desirable for a powder applicator to be able to achieve a high flow rate that may be associated with a minimum internal dimension above the second threshold (i.e., a minimum internal dimension that is too large to stop powder flow even when the applied vibration is stopped). Accordingly, the inventors have appreciated that, in some embodiments, there may be benefits associated with a vibrational powder applicator that include a valve and/or flow restrictor to prevent the free flow of powder through the nozzle and outlet of an applicator in the absence of vibrations being applied to the applicator by the associated one or more actuators. Specific embodiments are explained in greater detail below.

As noted above, in some embodiments, an applicator may include a flow restrictor. A flow restrictor may be a passive control structure that is configured to permit flow of powder when the actuator is activated, and prevent flow of powder when the actuator is deactivated. In one such embodiment, a flow restrictor may correspond to a body that is positioned within an interior volume of the applicator, such as within a powder storage chamber and/or a nozzle of the applicator. The body may reduce the open area through which the powder may flow by forming one or more gaps between the body and an interior surface of the applicator that the powder may flow through. The inclusion of these one or more gaps which may have a reduced characteristic dimension relative to the unobstructed nozzle and/or outlet may prevent the free flow of powder from the applicator when the one or more actuators are not activated. However, embodiments in which a flow restrictor is not used are also contemplated.

In some embodiments, a flow restrictor may include a body that is positioned at least partially within a chamber and/or nozzle of the applicator in which the powder may be disposed. The flow restrictor may form one or more gaps between an interior surface of the chamber and/or nozzle of a vibrational powder applicator and the body, such that the powder flows through the one or more gaps past the flow restrictor when vibrations are applied to the applicator by the associated actuator. In some embodiments, the flow restrictor may include a plurality of fins that extend outwards from the body towards an adjacent interior surface of the chamber and/or nozzle the body is disposed within, where the plurality of fins are configured to prevent flow of the powder when the actuator is deactivated. Without wishing to be bound by theory, the frictional and/or shear forces exerted on the powder by the fins may be sufficient to stop powder flow when vibrational energy is no longer applied to the applicator. In some embodiments, the plurality of fins of the flow restrictor may extend radially from the body of the flow restrictor where the body may be centrally located within the corresponding chamber and/or nozzle in some embodiments. The fins may extend fully toward an interior surface of the housing such that the fins contact the interior surface, or the fins may only extend partially toward the interior surface of the housing. In embodiments in which the fins extend fully toward the interior surface of the housing, the one or more gaps may be defined by surfaces including the interior surface of the housing, the side surfaces of the fins, and/or a surface associated with the body. In such embodiments, the fins may separate adjacent gaps from one another. In some embodiments, the fins may extend fully along a longitudinal dimension of the body. In some embodiments, the fins may be disposed on a proximal portion of the body. Thought in other embodiments, the fins may be disposed on a distal portion of the body or both a proximal and distal portion of the body as the disclosure is not limited to where or how a body used to restrict flow through the nozzle and outlet of an applicator includes fins. Additionally, embodiments in which fins are not used are also contemplated.

As described above, a flow restrictor may be a passive component in some embodiments. As such, a flow restrictor may be unpowered and/or unactuated. Thus, in some embodiments, a flow restrictor may be entirely static, and free of any moving parts.

In some embodiments, a flow restrictor may be modular component that may be inserted into and/or removed from an applicator. In such embodiments, a flow restrictor may be replaced if it becomes damaged, or may be exchanged for a different flow restrictor with different characteristics. For example, a first flow restrictor configured for use with a first powder may be installed within an applicator when the first powder is used in the applicator. When the same applicator is used with a second powder, the first flow restrictor may be replaced with a second flow restrictor configured for use with the second powder. Accordingly, a single applicator may be configured to controllably deliver a wide range of powder particle sizes and/or densities.

Without wishing to be bound by theory, a flow rate of powder through a flow restrictor may depend at least in part on the powder properties (e.g., particle size, powder density) and flow restrictor properties. In embodiments in which a flow restrictor includes a body and a plurality of fins extending from the central body, parameters of the flow restrictor that may affect powder flow rate may include but are not limited to the size of the central body and the number of fins. By changing these (and other) parameters, different flow rates may be achieved. For example, a flow restrictor may be associated with powder flow rates of greater than or equal to 0.01 g/s, 0.05 g/s, 0.10 g/s, 0.25 g/s, or 0.50 g/s. A flow restrictor may also be associated with powder flow rates of less than or equal to 1.00 g/s, 0.50 g/s, 0.25 g/s, 0.10 g/s, or 0.05 g/s. Combinations of the above noted ranges are contemplated including, for example, powder flow rates greater than or equal to 0.01 g/s and less than or equal to 1.00 g/s. Of course, a flow restrictor may be associated with powder flow rates other than those specifically noted above, and it should be appreciated that the present disclosure is not limited to flow restrictors associated with any specific powder flow rates.

In some embodiments, an applicator may include a valve to selectively prevent the flow of powder through a nozzle and/or outlet of the applicator. The valve may be configured to selectively permit or prevent flow of the powder from the powder storage chamber to the outlet. In some embodiments, a valve may be powered and/or manually actuated, and may be described as an active flow control element. In some applications, a valve may include a selectively moveable gate, such as a spring-loaded sliding gate, configured to control the flow of powder. The gate may be configured to block powder flow in its default (e.g., unactuated) position, and may be moved to permit powder flow when activated by a user. For example, an aperture in a sliding gate may be configured to be aligned with a conduit between the powder storage chamber and the outlet when a user depresses a button, and a spring may be configured to return the sliding gate to its default position when the button is no longer depressed such that the aperture in the sliding gate is no longer aligned with the conduit. Though instances in which a spring biased valve are not used and/or instances in which a valve includes a gate that is displaced out of the flow path of the powder without the use of an aperture are also contemplated. A valve may be driven manually (e.g., when a user depresses a button) or automatically (e.g., by a solenoid valve, or other actuator, that is controlled by an associated processor). In some embodiments, valve control may be coupled to vibrational actuator control, such that opening or closing the valve may also activate or deactivate the vibrational actuator respectively. While a gate valve is described above and shown in the figures, in some embodiments, a valve may also include a mechanical door, a ball valve, a pinch valve, or any other appropriate valve capable of restricting the flow of powder through the nozzle and/or outlet of an applicator. Accordingly, it should be appreciated that any valve configured to selectively permit or prevent flow of powder may be used as the disclosure is not limited in this regard.

The applicators disclosed herein may be used to fluidize and dispense a wide range of therapeutic powders, with varied particle sizes and densities. The inventors have shown through testing that powders with relatively larger size and/or higher density powders may be used with the currently disclosed applicators. For example, the applicator may be configured to fluidize powders having an average particle size that is greater than or equal to 100 μm, 200 μm, 300 μm, and/or any other appropriate size. A powder may also have an average particle size that is less than or equal to 1000 μm, 900 μm, 800 μm, and/or any other appropriate size. Combinations of the above noted ranges are contemplated including, for example, an average particle size of a powder that is greater than or equal to 100 μm and less than or equal to 1000 μm, or an average particle size of a powder that is greater than or equal to 500 μm and less than or equal to 1000 μm. In some embodiments, a particle size of a powder may be used to refer to a maximum diameter or other maximum dimension of a powder, although other interpretations of particle size may be appropriate in other embodiments, and the disclosure is not limited in this regard. In addition to the above, in some embodiments, the applicator disclosed herein may enable the use of a combination of multiple types of powder particles, each consisting of similar or different particle properties, such as size, density, etc. In addition to the above, in some embodiments, the powders may have a Hausner ratio greater than 1.18, although it should be appreciated that the applicator may be configured to fluidize and dispense powders with a Hausner ratio below 1.18 as well. For example, a Hausner ratio of one or more powders contained within an applicator may be greater than or equal to 1.18, 1.2, 1.3, and/or any other appropriate ratio. Correspondingly, the Hausner ratio may be less than or equal to 1.4, 1.3, 1.2, and/or any other appropriate ratio. Combinations of the foregoing are contemplated including, for example, Hausner ratios between or equal to 1.18 and 1.4 though ratios both greater than and less than those noted above are also contemplated. Additionally, while specific particle sizes are given above, particles with sizes both greater than and less than those noted above are also contemplated as the disclosure is not so limited.

In some applications, it may be desirable for an applicator to be capable of fluidizing a powder contained therein when the applicator is in any of a number of different orientations. As such, it may be desirable to arrange a powder storage chamber of the applicator such that it is above an outlet of the applicator relative to a direction of gravity when the applicator is being used. For example, an applicator may be intended to be used while held with the outlet oriented at least partially vertically downward relative to the direction of gravity while the powder storage chamber is disposed at least partially above the outlet. In this way, powder from the powder storage chamber may flow toward the outlet under the influence of gravity when the powder is fluidized, such as from applied vibrational energy. To facilitate this positioning of the powder, in some embodiments it may be advantageous to angle a longitudinal axis of the applicator relative to a horizontal axis (where a horizontal axis is perpendicular to a vertical axis that is parallel to the local direction of gravity). Angling the applicator may help to maintain the powder in a desired portion of the chamber. Appropriate angles of the applicator while in use may be greater than or equal to 10°, 20°, 30°, 40°, 50°, 60°, 70°, 80°, and/or any other appropriate angle (wherein 0° corresponds to a longitudinal axis of the applicator aligned with a horizontal axis, and wherein 90° corresponds to the longitudinal axis of the applicator aligned with the vertical axis (i.e., aligned with the local direction of gravity)). Appropriate angles of the applicator while in use may also be less than or equal to 90°, 80°, 70°, 60°, 50°, 40°, 30°, 20° and/or any other appropriate angle. Combinations of foregoing are contemplated including, for example, an applicator angle that is between or equal to 10° and 90° during use.

It should be understood that an applicator may have any appropriately shaped chamber for containing a powder to be dispensed. However, in certain embodiments, a chamber of an applicator may have an elongated shape with a longitudinal axis extending along a length of the chamber. For example, the chamber may generally be cylindrical in shape with hemispherical and/or rounded ends. Such a shape may facilitate fluidization and dispensing of the powder through an outlet. For example, the shape may be absent of any sharp edges, corners, and the like which may disrupt the fluidization of a powder within the chamber. However, embodiments in which sharp edges, corners, and other abrupt non-continuous design features are present along a flow path and/or within a chamber of an applicator are also contemplated as the disclosure is not so limited. For example a dog leg, or other sharp bend, may be present along a flow path connecting the various flow channels and/or chambers with one another.

The applicators described herein may be used to dispense any appropriate type of powder as the disclosure is not limited in this fashion. However, as noted above, in some embodiments, the various embodiments of powder applicators described herein may be used to dispense a powder including one or more therapeutic compounds which may also be referred to as a therapeutic powder. Therapeutic compounds for purposes of this application may correspond to any appropriate material including, but not limited to, any drug, medication, pharmaceutical preparation, contrast agent, and/or biologic such as a protein, antisense molecule, and gene therapy viral vector as the disclosure is not so limited. In a specific embodiment, the therapeutic compound may be a hemostatic agent in the form of a hemostatic powder. The amounts of therapeutic powder dispensed from an applicator may be selected such that an effective amount of the therapeutic compound may be dispensed at a desired location. When a therapeutic compound is present in a particular location in an “effective amount” it means a concentration of the therapeutic compound is greater than or equal to a trace amount and is sufficient for achieving a desired purpose, such as, for example, to permit detection of the therapeutic compound in a subject for diagnostic purposes, to treat a disease or condition in a subject, and/or enhance a treatment of a disease or condition in a subject. In some embodiments, an effective amount of a particular therapeutic compound is present in an amount sufficient to reduce or alleviate one or more conditions associated with a particular condition.

In some embodiments, a method of operating a vibrational powder applicator may include controlling a flow of pressurized gas. Pressurized gas may be used in addition to (or as an alternative to) vibrational energy to assist in fluidizing the powder and allowing the powder to flow from the powder storage chamber toward the nozzle. Flowing gas through a portion of the applicator (e.g., a portion of the applicator near or including the powder storage chamber) may entrain powder from the powder storage chamber in a gas flow for delivery through the nozzle. For example, a pressurized gas source may be in fluid communication with the outlet of the applicator, such that pressurized gas may flow from the pressurized gas source, through the applicator, and out of the outlet. Between the pressurized gas source and the outlet, the gas flow may entrain powder. For example, the gas flow may be routed past or through the powder storage chamber, such that powder may be entrained by the gas flow. An applicator may be configured to effectively control the delivery area of the powder by controlling a pressure and/or velocity of the gas flow. In some embodiments, a pressurized gas flow may be delivered manually or automatically. Appropriate pressure sources may include, but are not limited to, compressible bellows, a gas canister, a centralized pressure source such as a pressurized gas port, a pump, and/or any other appropriate pressure source capable of providing a pressurized gas to an applicator.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

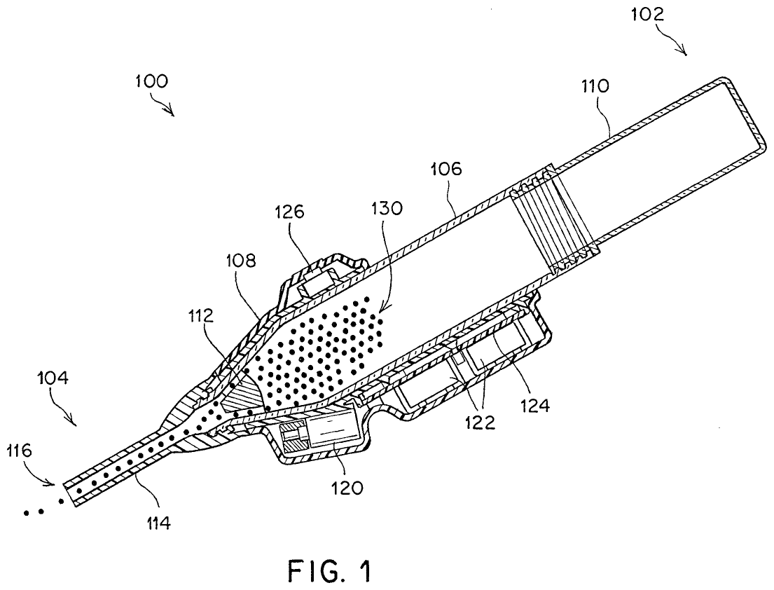

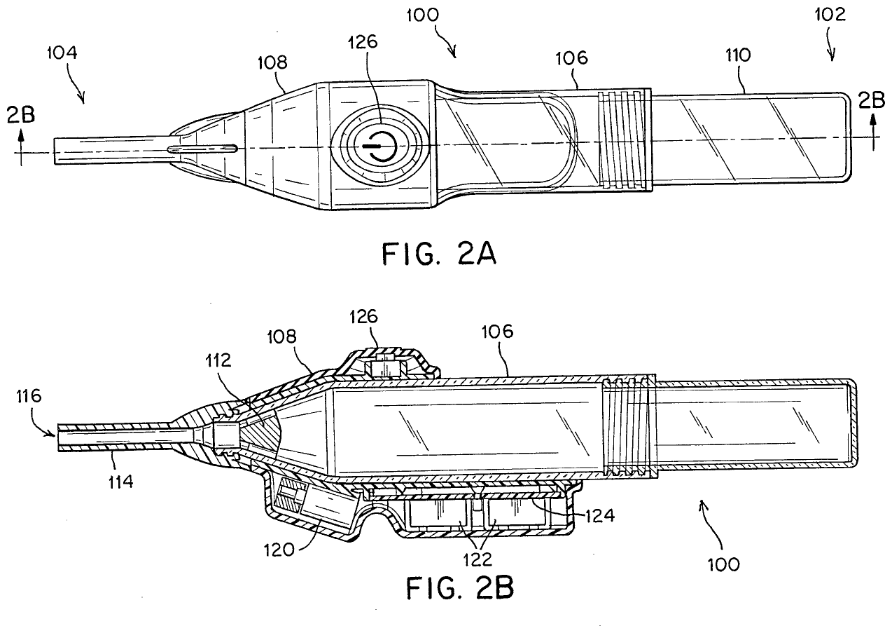

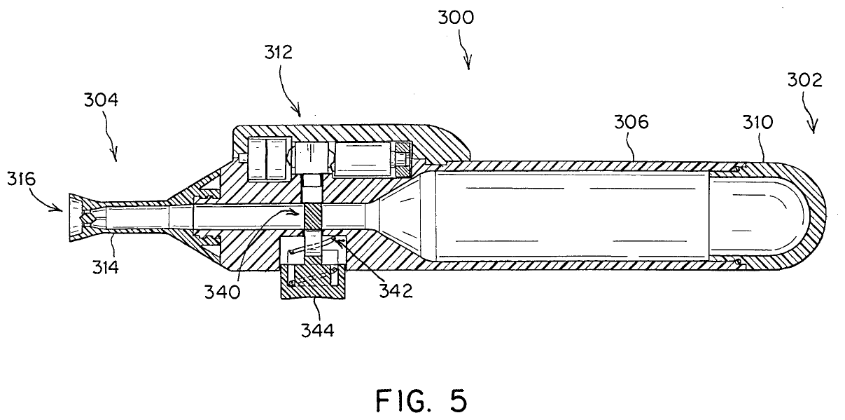

FIGS. 1-2B depict one embodiment of a vibrational powder applicator 100. FIG. 1 is a cross-sectional schematic of the applicator 100. FIG. 2A is a top view of the vibrational powder applicator 100, and FIG. 2B is a cross-sectional side view of the vibrational powder applicator 100. In this embodiment, the applicator 100 includes a proximal end 102 associated with a powder storage chamber 110 and a distal end 104 associated with an outlet 116 formed on a distal portion of the applicator. The powder storage chamber 110 is configured to store a powder 130 disposed therein, which may include a therapeutic powder such as a hemostatic powder or other appropriate powder. The powder storage chamber 110 may be operatively coupled to a housing 106 of the applicator 100. In some embodiments, the storage chamber 110 may be removably coupled to the housing 106 (e.g., using a threaded interface, fasteners, press fits, or any other suitable coupling). In some embodiments, the storage chamber 110 may be fixedly coupled to the housing 106 such that it may not be removed. In yet other embodiments, the chamber may be integrally formed in a portion of the housing such that the chamber is fully formed within the housing of the applicator. In the depicted embodiment, an overall powder storage chamber corresponding to the internal volume formed within the housing and the removable portion of the chamber may be provided. Regardless of the specific construction, the resulting powder storage chamber may include the powder disposed therein for subsequent dispensing by the applicator. A distal portion of the housing 106 may include one or more components to restrict the free flow of powder through the outlet of the applicator including, for example, a flow restrictor 112. Powder that flows through a gap between the one or more portions of the flow restrictor and the interior surface of the chamber and/or nozzle, or other portion of the housing, may flow through a nozzle 114 before exiting the applicator through the outlet 116. In some embodiments, an applicator 100 may include a sleeve 108 that surrounds at least a portion of the housing 106. The sleeve 108 may be configured to provide at least partial vibration isolation between the housing 106 and a user's hand. In some instances, the sleeve may also provide an ergonomic grip for the user. The sleeve 108 may additionally house driving components, such as an actuator 120, one or more batteries 122, a PCB 124, and/or an activation button 126. As discussed above, the actuator 120 may be configured to vibrate the powder 130 to fluidize the powder and allow the powder to flow out of the outlet 116. For example, the actuator 120 may include a motor configured to rotate an eccentric mass. In some embodiments, the button 126 may be configured to activate the actuator 120. In some embodiments, the button 126 may be configured to actuate both the actuator 120 and an active flow control element, as described in greater detail below in reference to FIG. 5. It should be understood that while the one or more actuators of the applicator have been shown as being disposed between the outer sleeve and applicator in the depicted embodiment, other constructions are contemplated. For example, the actuator may be operatively coupled with the housing of the applicator in any appropriate fashion including both direct, and indirect, connections with any desired portion of the applicator housing such that the one or more actuators are capable of applying vibrations to the housing of the applicator.

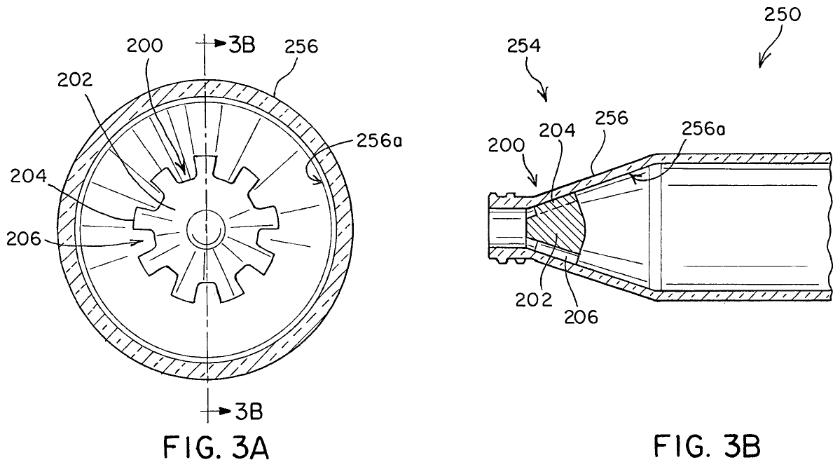

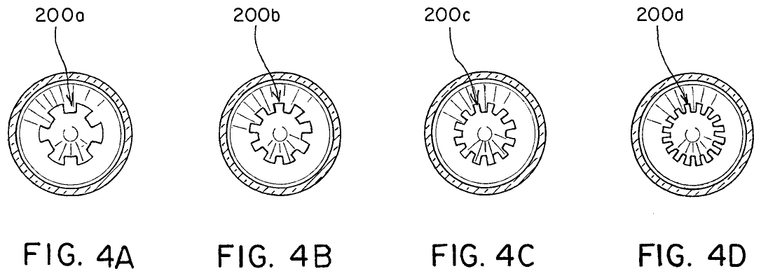

FIGS. 3A-4D depict different embodiments of a flow restrictor 200. FIG. 3A is a cross-sectional back view of one embodiment of a flow restrictor 200. The flow restrictor 200 may include a body 202 disposed in a central portion of the chamber and flow path and a plurality of fins 204 extending from the central body 202. However, embodiments in which the body is not disposed in a central portion of the chamber are also contemplated. In the embodiment of the figure, the fins 204 extend radially outwards from the central body 202. Depending on the embodiment, the fins may either have a gap between the fins and the interior surface 256a of the housing 256 of the applicator 250 or the fins may contact the interior surface of the housing. In either case, the fins 204 form at least one, and in some instances, a plurality of gaps 206 between the flow restrictor and the interior surface of the powder storage chamber formed in the housing to allow flow of the powder past the flow restrictor to the nozzle and outlet when the powder is fluidized.

Note that in the back view of FIG. 3A, it may be difficult to see the interface of the fins 204 with the interior surface 256a of the housing 256 due to the tapered geometry of a portion of the distal end 254 of the applicator 250. FIG. 3B is a cross-sectional side view of a distal end 254 of one embodiment of a vibrational powder applicator 250 that shows where a flow restrictor 200 may be disposed, taken along line 3B-3B shown in FIG. 3A. Consistent with FIG. 3A, FIG. 3B shows a fin 204 along a top center of the flow restrictor 200 and a gap 206 along a bottom center of the flow restrictor 200. As can be seen in FIG. 3B, the fin 204 extends up to and contacts the interior surface 256a of the housing 256 of the applicator 250. However, as noted above, embodiments in which the one or more fins do not contact the interior surface of the housing are also contemplated.

FIGS. 4A-4D are cross-sectional views of different embodiments of flow restrictors with different numbers of fins. FIG. 4A shows an embodiment of a flow restrictor 200a that includes six fins. FIG. 4B shows an embodiment of a flow restrictor 200b that includes nine fins. FIG. 4C shows an embodiment of a flow restrictor 200c that includes twelve fins. FIG. 4D shows an embodiment of a flow restrictor 200d that includes sixteen fins. While four specific examples of flow restrictors with different numbers of fins have been provided in FIGS. 4A-4D, it should be appreciated that a flow restrictor 200 may include any suitable number of fins, as the disclosure is not limited in this regard. Additionally, without wishing to be bound by theory, fewer fins may permit the passage of larger powder particles for the same gap size between the corresponding body and interior surface of the housing as compared to a larger number of fins which may permit correspondingly smaller particles to flow past the flow restrictor. Accordingly, the usage of different flow restrictors with either different gap sizes and/or numbers of fins may permit the usage of different size powders with the same overall applicator design.

FIG. 5 is a cross-sectional side view of one embodiment of a vibrational powder applicator 300 that includes a valve 312 configured to restrict the flow of powder through the applicator when it is in the closed configuration. For example, this may be desirable when some amount of powder may leak out from the device when oriented vertically downwards either due to powder size, outlet size, or other appropriate considerations. In the depicted embodiment, the applicator 300 includes a proximal end 302 associated with a powder storage chamber 310 operatively coupled to a housing 306, and a distal end 304 associated with a nozzle 314 and an outlet 316. The valve 312 is disposed between the powder storage chamber 310 and the outlet 316. In the embodiment of the figure, the valve 312 includes a moveable gate 340 operatively coupled to a spring 342 and a button 344. When a user depresses the button 344, the gate 340 moves to align an aperture in the gate with a conduit through which the powder is configured to flow. When the user releases the button 344, the spring 342 returns the gate 340 to its default position in which the aperture is not aligned with the conduit, thereby preventing flow. Thus, the valve may selectively permit and prevent the flow of powder through the nozzle and outlet based on whether or not the valve is in the open or closed configuration. While a specific gate valve has been shown in the figure, it should be understood that any appropriate type of valve capable of selectively restricting the flow of a powder through the nozzle and outlet of an applicator may be used as mentioned previously as the disclosure is not limited in this fashion.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

The present disclosure at times uses terms denoting relative positions such as “below” and/or “above”. It should be appreciated that these relative terms are used relative to a local direction of gravity. For example, a first object is understood to be “below” a second object if the second object moves toward the first object along a gravitational axis when acted upon solely by a gravitational force. It should be appreciated that an object that is “above” or “below” another object need not be “directly above” or “directly below” the other object. For example, if the local direction of gravity is aligned with a vertical direction, one object may be offset horizontally (i.e., perpendicularly from the gravitational axis) from another object and still be “above” or “below” the other object.

1. A method of applying a therapeutic powder, the method comprising:
positioning an outlet of a powder applicator containing a therapeutic powder below a powder storage chamber of the powder applicator relative to a local direction of gravity;
vibrationally agitating the therapeutic powder; and
dispensing at least a portion of the therapeutic powder through the outlet of the powder applicator.

2. The method of claim 1, further comprising positioning the outlet of the powder applicator above a target delivery site.

3. The method of claim 1, wherein agitating the therapeutic powder includes rotating an eccentric load.

4. The method of claim 1, wherein agitating the therapeutic powder includes activating an actuator.

5. The method of claim 4, wherein the actuator is contained within an outer casing of the powder applicator.

6. The method of claim 4, further comprising deactivating the actuator to stop dispensing the therapeutic powder.

7. The method of claim 1, wherein the therapeutic powder is a hemostatic powder.

8. A vibrational powder applicator comprising:
a powder storage chamber;
a therapeutic powder disposed within the powder storage chamber;
an actuator operatively coupled to the powder storage chamber and configured to vibrationally agitate the therapeutic powder when activated; and
an outlet in fluid communication with the powder storage chamber.

9. The vibrational powder applicator of claim 8, wherein the therapeutic powder is a hemostatic powder.

10. The vibrational powder applicator of claim 8, wherein the powder storage chamber and the outlet are configured such that the therapeutic powder moves from the powder storage chamber toward the outlet when the therapeutic powder is vibrationally agitated by the actuator.

11. The vibrational powder applicator of claim 8, wherein the actuator includes a motor coupled to an eccentric load.

12. The vibrational powder applicator of claim 8, wherein the actuator is contained within an outer casing of the powder applicator.

13. The vibrational powder applicator of claim 8, wherein a diameter of the therapeutic powder is greater than or equal to 100 μm and less than or equal to 1000 μm.

14. The vibrational powder applicator of claim 13, wherein a diameter of the therapeutic powder is greater than or equal to 500 μm and less than or equal to 1000 μm.

15. The vibrational powder applicator of claim 8, wherein the applicator includes a proximal end and a distal end, wherein the outlet is positioned on a distal portion of the applicator, and wherein the actuator is disposed proximally relative to a distal portion of the powder storage chamber.

16. The vibrational powder applicator of claim 8, wherein the applicator includes a proximal end and a distal end, wherein the outlet is positioned on a distal portion of the applicator, and wherein the actuator is disposed distally relative to a proximal portion of the powder storage chamber.

17. A vibrational powder applicator comprising:
a powder storage chamber configured to contain a powder;
an actuator operatively coupled to the powder storage chamber and configured to vibrationally agitate the powder when activated;
an outlet in fluid communication with the powder storage chamber; and
a flow restrictor disposed between the powder storage chamber and the outlet.

18-20. (canceled)

21. The vibrational powder applicator of claim 17, wherein the flow restrictor comprises a body that forms one or more gaps between an interior surface of a housing of the vibrational powder applicator and the body, wherein the one or more gaps are configured such that the powder flows through the one or more gaps when the actuator is activated.

22-29. (canceled)

30. A vibrational powder applicator comprising:
a powder storage chamber configured to contain a powder;
an actuator operatively coupled to the powder storage chamber and configured to vibrationally agitate the powder when activated;
an outlet in fluid communication with the powder storage chamber; and
a valve disposed between the powder storage chamber and the outlet, the valve configured to selectively permit or prevent flow of the powder from the powder storage chamber to the outlet.

31-33. (canceled)

34. The vibrational powder applicator of claim 30, wherein the valve comprises a selectively moveable gate.

35. The vibrational powder applicator of claim 34, wherein the selectively moveable gate is configured to control the flow of the powder.

36-40. (canceled)
